(19) United States
(12) Patent Application Publication
(10) Pub. No.: US 2016/0222349 A1
(43) Pub. Date: Aug. 4, 2016

(54) DIFFERENTIATION OF RPE PROGENITORS INTO NEURON-LIKE CELLS

(71) Applicant: Stemedica Cell Technologies, Inc., San Diego, CA (US)

(72) Inventor: Ludmilla Kharazi, San Diego, CA (US)

(73) Assignee: Stemedica Cell Technologies, Inc., San Diego, CA (US)

(21) Appl. No.: 15/084,018

(22) Filed: Mar. 29, 2016

Related U.S. Application Data
(60) Provisional application No. 62/109,266, filed on Jan. 29, 2015.

(51) Int. Cl. C12N 5/0793

(52) U.S. Cl. CPC C12N 5/0619; C12N 2506/08; C12N 2501/115

(57) ABSTRACT

A method of permanently culturing RPE cells under hypoxia, and differentiating the RPE cells into neuronal like cells.

This application takes priority from provisional patent application Ser. No. 62/109,266, filed Jan. 29, 2015.

### BACKGROUND

Retinal pigment epithelium is a monolayer of polarized and pigmented cells (RPE cells) that are connected to each other by tight junctions. The tight junctions form an important part of the barrier function of the whole blood retinal barrier. RPE is located between the photoreceptor cells of the retina and choroid. This orientation is necessary for the RPE layer to perform many of its functions including photo pigment recycling, ion and fluid transport, phagocytosis, and release of various factors that protect the integrity of the outer retina and choriocappillaris. One of the therapeutic options is to use RPE cell cultures for cell replacement therapy.

Oxygen concentration in cell culture plays an important role in regulating RPE cellular functions. Hypoxia prevents the cells' senescence and increases cell proliferation and response to exogenous factors. Hypoxia induces the gene expression of a variety of proteins such as erythropoetin, VEGF and glucose transporters. However, the role of low oxygen in RPE cell differentiation and barrier properties in vitro remains to be investigated.

### SUMMARY

The invention involves a method for differentiating retinal pigmented epithelium (RPE) into neuronal-like cells comprising the step of culturing RPE cells in vitro under low oxygen. Promoting the method is supplementation of the culture with bFGF. Preferably the method involves permanent culturing under hypoxia, which excludes any culturing under non-hypoxic conditions.

### BRIEF DESCRIPTION OF FIGURES

FIG. 1. Hypoxia and growth factors stimulate RPE cell proliferation.

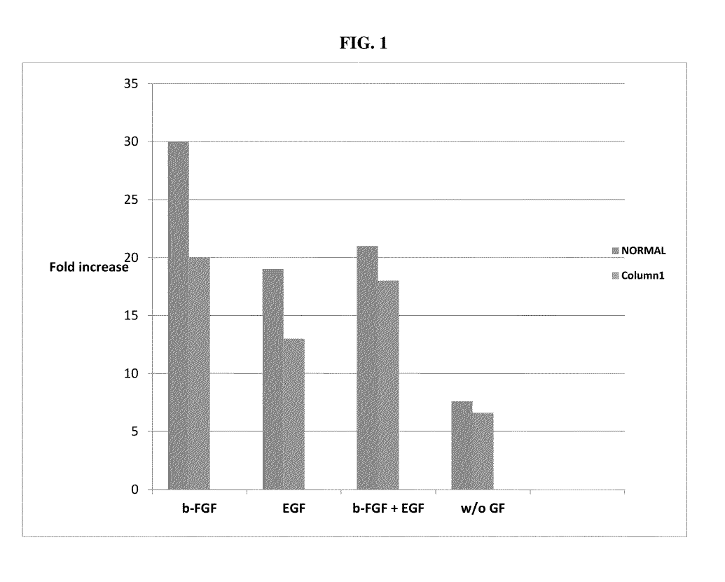

FIG. 2. Changes of TER during RPE differentiation (seeding density is 1×105 per insert). The TER of the cell monolayers cultured in normal oxygen reached maximum after 8 weeks. The cells cultured in low oxygen maintained low TER. Hypoxia-grown RPE progenitors differentiated under normal O2 developed and maintained higher TER compared to normoxia-grown cells differentiated under the same conditions.

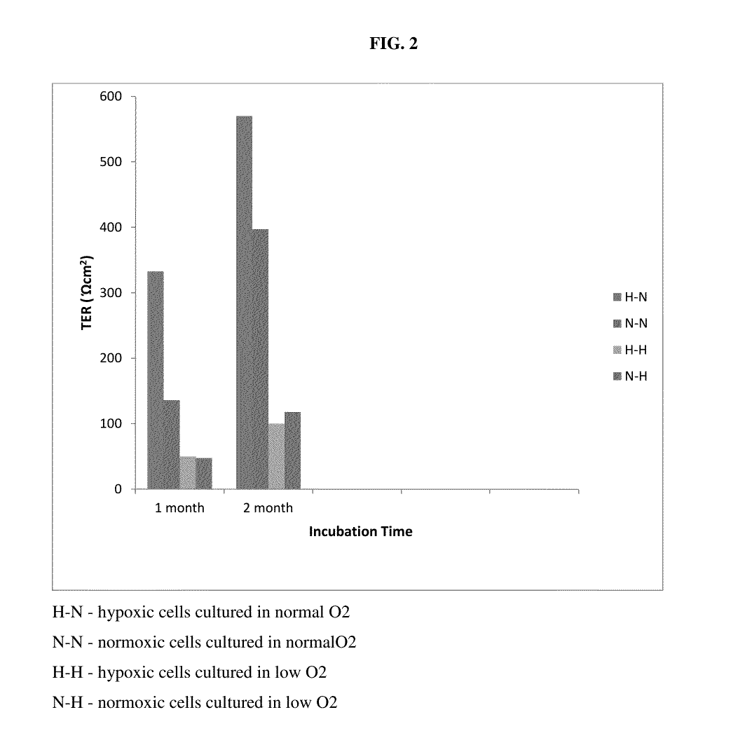

FIG. 3. Morphological and immunocytochemical characterization of hypoxic RPE progenitors differentiated under low O2 conditions after 10 days of culturing. (100×).

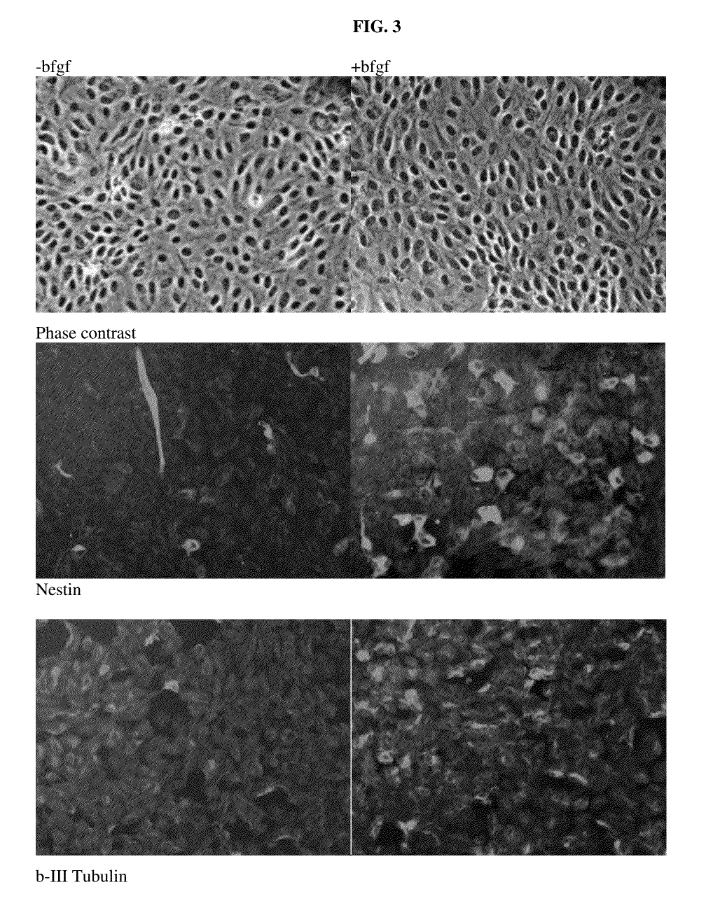

FIG. 4. Morphological and immunocytochemical characterization of hypoxic RPE progenitors differentiated under normal O2 conditions (100×).

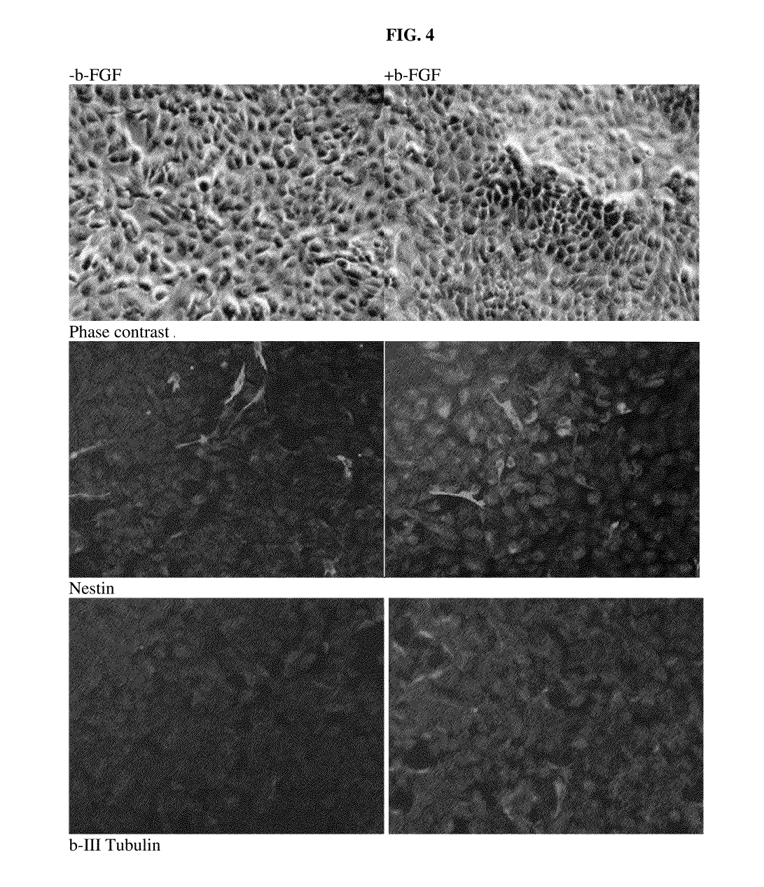

FIG. 5. Morphological and immunocytochemical characterization of hypoxic RPE progenitors differentiated under low O2 conditions after 20 days in culturing. (100×).

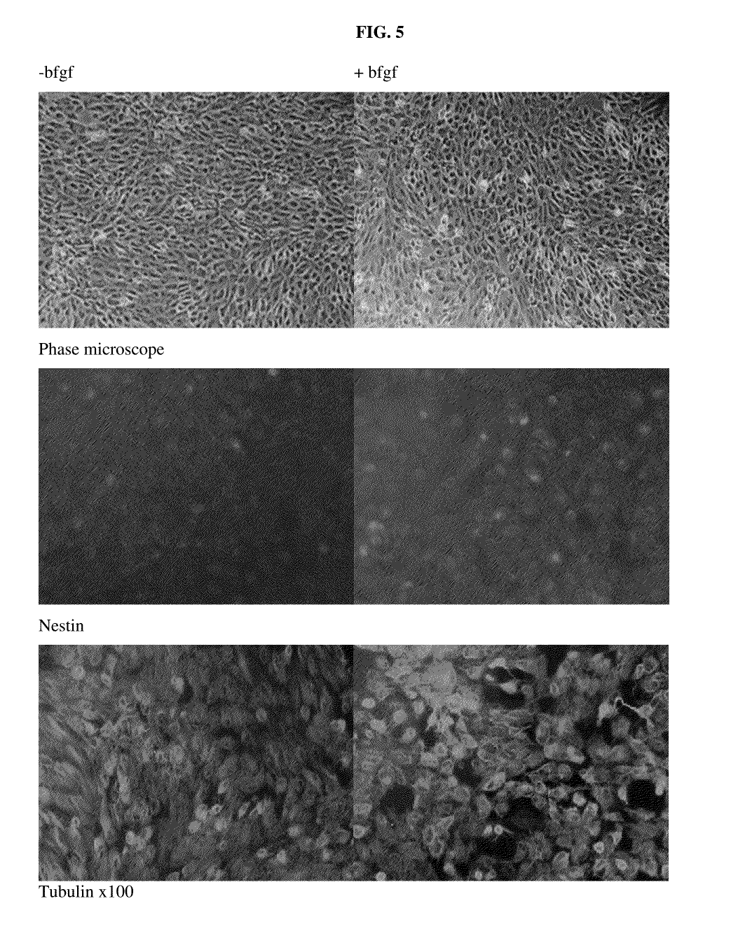

FIG. 6. Morphological and immunocytochemical characterization of hypoxic RPE progenitors differentiated under normoxic conditions. 20 days in culture. (100×).

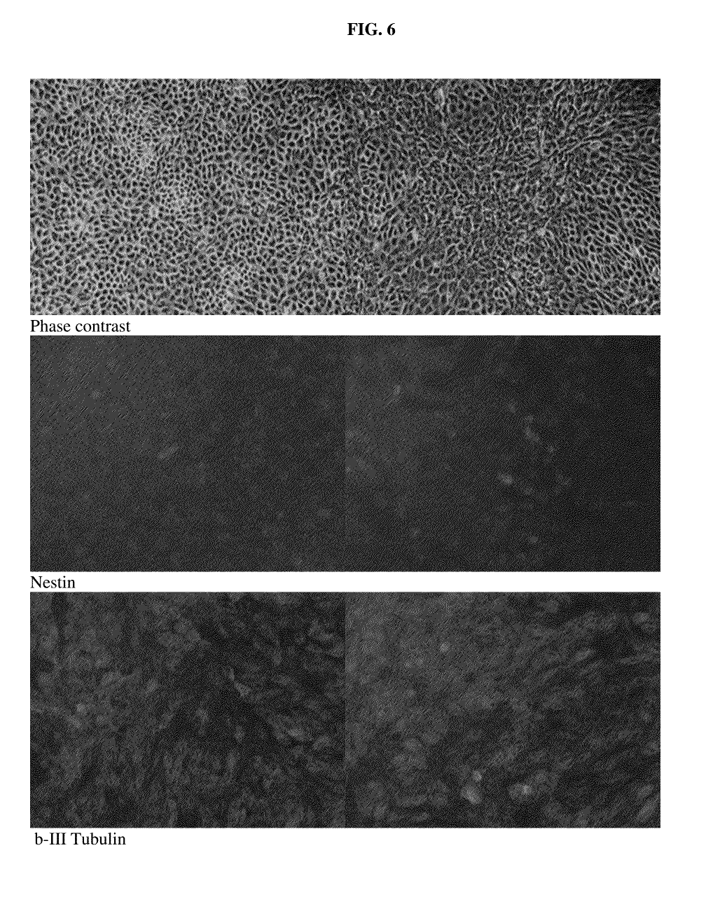

### DETAILED DESCRIPTION

The present invention embodies a method of differentiating RPE cells in vitro into neuronal-like cells. In this study the role of environmental oxygen concentration on the induction of neuronal markers during maturation of RPE progenitors in tissue culture is illustrated.

Human fetal eyes (18 weeks gestation age) were obtained from Advanced Bioscience Recourses (ABR) Inc. Primary cells were propagated in low (5%) O2 conditions in MEM medium containing 5% of FBS until passage 3. The expression of RPE specific markers was tested by flow cytometry. For differentiation purposes, expanded RPE progenitors were cultivated in transwell permeable inserts under normal or low O2 conditions in RPE medium containing 1% FBS. Barrier function was assessed by measuring transepithelial resistance (TER). The cell morphology and expression of neuronal markers such as beta-III tubulin and RPE markers such as ZO1 and Na/K ATPase were evaluated by immunocytochemistry.

Prior to differentiation, RPE progenitors were ≧90% positive for pancytokeratin, ZO1, and RPE65. As reported previously, RPE progenitors differentiated under normal oxygen conditions showed signs of mature RPE cells such as increased TER, hexagonal morphology, re-pigmentation, well-developed tight junctions, clear presence of ZO-1 and Na/K ATPase. The expression of beta III tubulin was insignificant or not detectable. In contrast, RPE cells differentiated in low oxygen did not upregulate the TER values. The cells appeared heterogeneous with no signs of pigmentation and some of them morphologically resembled neural cells. On day 10 in tissue culture, cells showed high level of nestin and low beta III tubulin expression. On day 20, however, beta-III tubulin marker was acquired while nestin expression was down regulated. These data suggest that low oxygen concentration promotes induction of neuron-like cells from RPE progenitors during their maturation in vitro.

The phrases “low oxygen conditions,” “low oxygen,” “reduced oxygen tension,” and “hypoxia” as used herein refer to any oxygen concentration that is less than atmospheric oxygen. Low oxygen conditions include, but are not limited to, less than about 20%, 19%, 18%, 17%, 16%, 15%, 14%, 13%, 12%, 11%, 10%, 9%, 8%, 7%, 6%, 5%, 4%, 3%, 2%, or 1% oxygen.

### EXAMPLES

### Materials and Methods

Cell Culture:

Human fetal eyes (18 weeks gestation age) were acquired from Advanced Bioscience Recourses (ABR) Inc. The cornea, lens and retina were removed and the eyecup dissected into 4 quadrants was treated with 2% dispase solution at 37° C. for 30 min in the 37.0 incubator. The RPE cells were isolated from choroid as intact sheets under dissection microscope.

The cells were propagated in 9 cm2 tissue culture dishes either under normal (20%) or low (5%) O2 conditions at a density of 1.5×103 cells per cm2 in MEM Alpha Modification medium supplemented with 5% FBS, GlutaMax, and Nonessential Amino Acids and growth factors until passage 3.

Flow Cytometry.

Purity of RPE cells was assessed by flow cytometry to determine the percentage of the hRPE population expressing RPE65 and epithelial and fibroblast proteins.

RPE Monolayers.

Monolayers of both types were grown on permeable membrane fibronectin-coated filters. Barrier functions and were assessed by transepithelial resistance (TER) measurements. To investigate and compare the characteristics of RPE progenitors and their ability to express neural properties, cells were cultured in fibronectin-coated 24-well plates for 10 and 20 days in media containing 1% FBS with or without b-FGF growth factor. Expressing of specific peripheral membrane protein ZO-1, and neural progenitor markers Nestin and the young neuronal marker beta III-tubulin were examined using immunocytochemistry.

Immunocytochemistry.

RPE cells were first fixed by 2% paraformaldehyde for 30 minutes at room temperature. Then the cultures were washed three times in PBS at room temperature. The cells were made permeable using 0.2% Triton X-100 (VWR, West Chester) in PBS for 30 minutes and cultures were blocked in 1% bovine serum albumin for 60 minutes at room temperature. Rabbit anti ZO-1 (1:100, Life Technologies) and Anti-Na/K/ATPase a-1 monoclonal mouse antibody (1:100, Millipore) against specific membrane proteins were used to confirm RPE characteristics. The cultures with primary antibody were incubated for 1 hour at room temperature (negative controls were incubated with PBS only). The cultures were washed in PBS three timed before incubation with secondary antibodies. Fluorescent green anti-mouse IgG and anti-rabbit IgG (1:500, Vector) were used as secondary antibodies. The cultures were examined by Zeiss fluorescent microscope.

Conclusions:

B-FGF substantially stimulates RPE cell proliferation in 5% FBS MEM media on plastic dishes in low O₂ environment.

O2 is required for RPE progenitor cells differentiation in vitro.

RPE progenitor cells grown permanently in hypoxia have a greater differentiation potential compared to normoxic cells.

The RPE cells show potential to de-differentiate in low O₂ environment into a cell type with neuronal-like identity.

Hypoxia promotes induction of neuron-like cells from RPE progenitors during their maturation in vitro. b-FGF potentiates this effect.

RPE Cells Process Flow (P0-P3) for Fetus of 17 Week Gestation

| Process | Yield |
|---|---|
| Tissue Processing | N/A |
| P0 | 1.2 × 10⁶-3.0 × 10⁶ |
| Plate 1 mL freshly isolated fetal RPE suspension containing | |
| the cells and pieces of the broken sheets in 15 cm TCDs | |
| Number of TCDs: 6 TCDs per2 eye balls | |
| Duration: 6-8 days in culture | |
| Harvest when islands of the small non pigmented tightly | |
| packed hexagonal cells are observed | |
| Expected yield for = 2.0 × 10\hat{ }5-5.0 × 10\hat{ }5 cells/1-15 cm | |
| TCD | |
| P1 (MCB) | 80 × 10⁶-250 × 10⁶ |
| Harvest P.0 | Freeze at 1 × 10⁶ |
| Seed at 8 × 10\hat{ }4/TCD/10 mL media | 80-250 vials |
| Number of TCDs: 15-40 | (MCB) |
| Seeding density: 1.3 × 10³/cm₂; | |
| Duration: 3-5 days in culture | |
| Change media every 2-3 days | |
| Harvest at 80%-85% confluence | |
| Expected yield = 6 × 10⁶-7 × 10⁶ cells/TCD | |
| P2 | 25 × 10\hat{ }6-40 × 10\hat{ }6 |
| Thaw 1 MCB vial; assume 0.5-0.7 × 10\hat{ }6 cells upon | Do not freeze. Use for |
| recovery; | seeding of P2 in |
| Seed at 1 x 10⁵/TCD/10 mL media (5-7 9 cm TCDs) | 636 cm₂ Corning Chambers |
| Seeding density: 1.6 × 10³/cm²; | |
| 3-5 days in culture; change media every 2-3 days | |
| Harvest at 80%-85% confluence | |
| Expected yield = 5 × 10⁶-7 × 10⁶ cells/TCD | |
| P3 FP | 500 × 10⁶-1000 × 10⁶ |
| Seed at 2 × 10⁶/636 cm₂ Culture Corning Chamber | Freeze |
| Seeding density: 0.3 × 10³/cm2 | 2 × 10⁶ |
| Number chambers: 10-20 | 250-500 vials or |
| 3-6 days in culture | 1 × 10\hat{ }6-1000 |
| Change media every 2-3 days | vials |
| Harvest at 80%-85% confluence | FP |
| Expected Yield = 50 × 10⁶-60 × 10⁶ cells/636 cm₂ Corning Cell | |
| Culture Chamber | |
| Creation of (P4) FP: 5 × 10⁶/1.8 mL vial | |

Total pop doubl 17-18

### Certified/Calibrated Equipment

| | | | PM and/or |
|---|---|---|---|
| Description | Supplier | Model # | Calibrated |
| Biosafety Cabinet | ThermoScientific | 1284 | Certified |
| 5532 Series Centrifuge | ThermoScientific | 5532-1L | Cal & PM |
| | | GP | |
| +4° C. Refrigerator | Thermo Scientific | 3773 | Cal & PM |
| Light Microscope | Fisher Micro Master | MC200 | PM |
| Inverted Microscope | Carl Zeiss | Axiovert | PM |
| Axiovert 25 | MicroImaging, Inc. | 25 | |
| −80° C. Freezer | Thermo Electon | 995 | Cal & PM |
| | Corporation/Forma | | |
| 37° C., 5% CO₂ | Thermo Scientific/Forma | 3110 | Cal & PM |
| Incubator | | | |
| 37° C. Water Bath | Labline instruments Inc. | 18802 | Cal |
| Harvard Trip Balance | OHAUS | N/A | N/A |
| (1400/1500 series) | | | |
| Control Rate Freezer | Thermo Scientific | 7450 | Cal & PM |
| Quarantine Storage | Taylor Wharton/WestAir | LS750B-R | Cal & PM |
| Liquid Nitrogen | Gases | | |
| Dewar | | | |
| Liquid Nitrogen Tank | WestAir | LN230-22 | Cal & PM |
| Timer (Fisher) | FisherScientific | 14-649-14 | |
| Drummond Pipetting | Drummond Scientific | N/A | N/A |
| Aid | Company | | |
| Dissecting microscope | | | |

### General Equipment & Materials

| | | | Stemedica |
|---|---|---|---|
| | | | Item |
| | | | Specifi- |
| Description | Supplier | Catalogue # | cation # |
| 5 mL Pipette | Fisherbrand | 13-678-11D | IS-2060 |
| 10 mL Pipette | Fisherbrand | 13-678-11E | IS-2061 |
| 50 mL Pipette | Fisherbrand | 13-678-11F | IS-2063 |
| 100 mL Pipette | Corning | 4491 | IS-2064 |
| Tissue Culture Dish | BD Falcon/Fisher | 353053 | NEW |
| (15 cm) | | | |
| Corning 1-Stack Cell | Fisher | 12-565-25 | NEW |
| Culture Chamber | | | |
| (636 cm²) | | | |
| Razor blade | Ted Pella | 121-3 | NEW |
| Clear Cut Sideport Knife | Cornstone Alliance | Alcon | NEW |
| | Navigator | 8065921540 | |
| # 5 Dumont Forceps | Kent Scientific | INS14096 | NEW |
| | Corporation | | |
| Test Tubes, 5 mL | BD Falcon/Fisher | 14-959-6 | IS-2047 |
| 50 mL centrifuge tubes | BD Falcon/Fisher | 14-959-49A | IS-2049 |
| 225 mL centrifuge tubes | BD Falcon/Fisher | 05-538-61 | IS-2050 |
| Cryo Tube, Vial, 1.8 mL | Nunc/Fisher | 12-565-167N | IS-2051 |

### Media Formulation and Solutions

PBS-GENTAMICIN/AMPHOTERICIN SOLUTION (500 mL)

| Name | Quantity | Units | Manufacturer |
|---|---|---|---|
| PBS | 499 | mL | Gibco |
| Centamicin/Amphotericin Solution | 1 | mL | Life Technologies |
| 500X | | | |

RPE Growth Media (500 mL)

| Name | Quantity | | Manufacturer |
|---|---|---|---|
| MEM | 462 | mL | Sigma |
| NEAA | 5 | mL | Sigma |
| GlutaMax | 5 | mL | Invitrogen |
| bFGF | 10 | μg | PeproTech |
| Heparin Sodium InJ.1,000 USP/mL | 3 | mL | APP |
| | | | Pharmaceutical |
| FBS | 25 | mL | Thermo-Sci |
| B-27 supplement (50X) minus | 10 | mL | Gibco |
| vitamin A | | | |

Complete Freezing Medium w/10% DMSO (500 mL)

| Name | Quantity | Units | Manufacturer |
|---|---|---|---|
| RPEGM | 450 | mL | In House Prep |
| DMSO | 50 | mL | Sigma |

2% Dispase Solution

| Name | Quantity | Units | Manufacturer |
|---|---|---|---|
| Dispase I | 0.2 g | mL | Sigma-Aldrich |
| HBSS w/o phenol red | 10 | mL | Invitrogen |

1% BSA solution

| Name | Quantity | | Units | Manufacturer |
|---|---|---|---|---|
| BSA | 0.1 | g | mL | |
| HBSS w/o phenol red | 100 | ml | mL | Invitrogen |

Donor Eligibility

1. Qualified medical personnel obtained patient informed consent. Human fetal eyes were obtained from Advanced Bioscience Resources (San Diego) with gestation age of 17 weeks and placed in a sterile plastic container pre-filled with 30 mL of RPMI (transport media) media containing Gentamicin/Amphotericin Solution (1 ml of Gentamicin/Amphotericin Solution B 500× add to 499 mL of RPMI). The containers packed on ice were shipped via a courier and fetal eyes were processed the same day of arrival.

2. The specimen was packaged and shipped on ice packs, informed consent was included with shipment.

Donor Characterization

Donor characterization required the following items:
i. General information
ii. Informed consent
iii. Donor's ID (MMDDYY-1) or as specified by clinic. Once received in house Donor Specimen received a number per Stemedica SOP in the form of DS-XXXX.
iv. The results of blood screening tests performed by Quest Diagnostics (San Diego, Calif.)
   1. HIV-1 and -2 tested by PCR
   2. HTLV-I and II tested by PCR
   3. HBV—tested by PCR
   4. HCV—tested by PCR
   5. CMV—tested by PCR
   6. EBV—tested by PCR
   7. Mycoplasma—tested by PCR
   8. Treponema pallidum—tested by PCR, or serologically

Isolation and Processing of Donor Specimen

The Donor Specimen (fetal eyes) arrived in sterile sample container in a transport media and received per item specification by Materials Management and assigned a DS-XXXX number. The specimen was transferred to ISO 7 Process Development Cleanroom 317 and stored in the 4° C. refrigerator until processing.

Isolation of Primary Human Fetal RPE Cells

1. All instruments sterilized using a steam autoclave.

2. Once the fetal eyes were received, the globes were placed into a 50 ml tube containing 30 ml PBS—Gentamicin/Amphotericin Solution and washed using hand rotation for 1 min.

3. The eye(s) were placed in a new 50 ml tube and wash the globe(s) using hand rotation for 1 min.

4. Above steps repeated 8 more times.

5. The eye(s) were placed in a new 50 ml tube pre-filled with PBS w/o antibiotics and the globe(s) were washed for 1 minute with 30 ml sterile PBS to wash away the antibiotics.

6. Repeat Step 5 one more time.

7. Removed one fetal eyeball #1 from the tube and placed into 9 cm sterile petri-dish.

8. Placed the tube with another eyeball #2 into a +4° C. refrigerator until used.

9. Using a dissecting microscope made an incision into the cornea of the eye ball #1 using a sterile razor blade (or sideport knife).

10. Placed the point of one blade of an iris scissor into the opening and carefully cut without high pressure the cornea/iris complex 1-2 mm posterior to the corneal limb and removed the cornea/iris tissue.

11. Removed the vitreous using a sterile inoculating loop. Gently held one edge of the eye cup with a pair of tweezers (#5 Dumont forceps) and used the loop to gently tease the vitreous out. The vitreous was typically removed intact.

12. Held one edge of the eye, dissected the globe into 4 quadrants with a razor blade in order for the eye cup to lay flat. The cut was made as close to the optic disc as possible.

13. Removed the sensory retina using an inoculating loop. Took care not to scratch the RPE layer.

14. Used one pair of tweezers (#5 Dumont forceps) to hold the edge of the dissected eye with another pair of tweezers gently peeled off the RPE/choroid layer from the sclera and placed into 2 ml of HBSS w/o phenol red in a well of the 6-well plate. RPE sheet when large was placed into the HBSS and when small is picked up with a sterile transfer pipette and placed into the HBSS.

15. Using transfer pipette gently picked up the RPE-choroid sheets and transferred to 2 ml of 2% dispase solution in a well of the 6-well plate. Incubated for 30 min at 37° C., with 5% CO₂.

16. Repeated steps 9-15 for another eyeball #2.

17. After dispase incubation of the sheets, gently transferred the RPE sheets with a sterile pipette to 2 ml of 1% BSA solution to stop the activity of the dispase. Kept the RPE sheet in 1% BSA solution until dissection was complete. Rinsing off the dispase was important in order to prevent excess digestion of the tissue and release of contaminating cells from the choroid.

18. Transferred the RPE sheet to a 10 cm sterile petri dish containing 1 ml of 1% BSA solution. Peeled the RPE layer from the choroid with fine forceps (Dumont forceps #5) in the % BSA solution. Placed the sheets in the new well of 6-well plate pre-filled with 2 ml of the of 1% BSA solution using forceps and sterile transfer pipet until all the RPE sheets were collected.

19. Repeated the steps 17-18 and for another eyeball.

20. Collected the sheets into a 15 ml centrifuge tube in 5-10 mL of 1% BSA solution.

21. By gently pipetting up and down 15-20 times, broke apart the RPE sheets using sterile 5 ml pipette.

22. Spun down RPE suspension at 500 RCF for 5 minutes, removed supernatant and re-suspended pellet in 6 mL of RPEGM.

23. Placed the 1 ml aliquot into 15 cm TCDs pre-filled with 19 ml of RPEGM (3 TCDs per one specimen with gestation age 17 weeks.

24. Changed the culture medium twice per week for 7-8 days.

Expansion of Passage 0.

The RPE cell production process began with the seeding of the cell suspension isolated from fetal eyes into 15 cm TCDs and the aseptic processing of the Passage 0 culture in Manufacturing Cleanroom.

Seeding P0.

1. When the 50 mL tube containing the RPE cell suspension was received, placed the tube into the BSA.

2. Placed 15 cm cell culture dishes to the BSC and pre-fill via pipet with 19 mL of pre-warmed RPEGM.

3. The RPE tissue/cell suspension from the isolation and processing of donor specimen

4. (Section 8) was mixed slowly by pipetting up and down 15-20 times with pipet to break apart the RPE sheets.

5. After mixing 1 mL of cell suspension transferred to each cell culture dish with a pipet.

6. After seeding was complete, gently mixed cell suspension in each dish using 10 mL pipet to achieve uniform distribution and transferred dishes to a 37° C., 5% CO2, 5% O2 incubator.

7. Monitored the dishes as required to check cell growth, change media twice a week.

8. RPE cells grew as sheets of non-pigmented, epitheloid hexagonal cells. Some cells of the fibroblastic morphology may have grown between the sheets of RPE cells. Before a day of harvest observed each dish and circled the areas of non-RPE cells using a permanent marker. Removed contaminating cells with a sterile transfer or 5 mL pipet. Changed the medium and placed into the incubator.

Harvest (P0) and Expansion to P1

After the culture was expanded for 7-8 days and cells formed dense areas of hexagonal tightly packed cells, the harvest was initiated.

1. Observed the marked areas in each dish. Removed remaining contaminating cells if found.

2. The media from each TCD was removed and discarded via pipette.

3. 10 mL of phosphate buffered saline (PBS) was added to each TCD via pipette. The TCDs were rocked back and forth to rinse the attached cell layer. The PBS from each TSD was removed and discarded.

4. 10 mL of TrypLE Express Solution was added to each TCD. The TCDs were rocked back and forth to ensure that the cells are saturated. The TCDs are incubated in at 37° C., 5% CO₂ and 5% O₂ for minimum 10 minutes.

5. At the completion of incubation the TCDs were removed from the incubator and observed under a microscope to ensure that a majority of the cells had detached.

6. 10 mL of RPEGM was added to each TCD via pipette. The dishes were washed by rinsing media over the TCD surface several times via pipette. When complete the cells and media were collected via pipette and transferred to the two of 225 mL tube(s) (one tube per two-three TCDs). This step is repeated a second time with 10-15 mL of warm HBSS to ensure that the majority of the cells have been collected.

7. The tubes were centrifuged at 500 RCF for 5 minutes at room temperature. At the completion of centrifugation the supernatant was discarded and the cells were re-suspended in 0.5-1.0 mL of RPEGM in each tube. The cells were collected to one tube.

8. The cells were mixed by gentle aspiration to ensure a homogeneous suspension. A cell count was performed using a hemacytometer.

9. The cell suspension was diluted to a target concentration of 8×10⁴/mL with RPEGM for seeding in 9 cm TCDs.

10. Calculated number of 9 cm TCDs were prepared by adding 9 mL of RPEGM via pipette to each one. Number of 9 cm TCDs to inoculate: Total number of cells/8×10̂4.

11. The cells were aspirated and 1 mL of cells was seeded via pipette into each dish.

12. The dishes rocked back and forth for even cell distribution.

13. The dishes were incubated at 37° C., 5% CO₂ and 5% O₂.

14. The dishes were monitored as required to check for confluence. The media was changed every 2-3 days

15. The culture was expanded for 3-5 days until the cells reached a confluence of 80-85%.

Harvest P.1 and Creation of MCB.

After the culture had been expanded for 3-6 days and the chambers had reached a confluence of 80-85%, the harvest was initiated. The cells were harvested and collected, counted, re-suspended in freezing medium and 1 mL was aliquoted into 1.8 mL cryovials at a target concentration of 1×10⁶ cells/mL. The stepwise procedure was as follows:

1. The media from each TSD was removed and discarded via pipette.

2. Next 7 mL of phosphate buffered solution (PBS) was added to each TCD via pipette. The TCDs were rocked back and forth to rinse the attached cell layer. The PBS from each culture vessel was removed and discarded via pipette.

3. Next 5 mL of TrypLE Express Solution was added to each TCD. The TCDs were incubated at 37° C., 5% CO₂ and 5% O₂ for a minimum of 10 minutes.

4. At the completion of incubation the culture vessels were removed from the incubator.

5. Next 5 mL of RPEGM was added to each TCD via pipette

6. When complete the cells and media were collected via pipette and transferred to the required number of 225 mL tubes (one tube per 5 TCDs). This step was repeated a second time by adding 10 mL of HBSS to each TCD to ensure that the majority of the cells had been collected.

7. The tubes were centrifuged at 500 RCF for 5 minutes at room temperature. At the completion of centrifugation the supernatant was discarded and the cells were re-suspended in 10 mL of RPEGM in each tube. The cells were aspirated and transferred to one collecting tube.

8. The cells were mixed by gentle aspiration to ensure a homogeneous suspension. A cell count was performed using a hemacytometer. Next, the tubes were centrifuged at 500 RCF for 5 minutes at room temperature. The supernatant was removed and discarded from the tube.

9. The cells diluted in freezing media to a target concentration of 1×10⁶ cells/mL. The cells were aspirated and 1 mL was aliqouted into 1.8 mL cryovials utilizing a 5 mL pipette. A maximum of 5 cryovials was aliquoted at one time, the cells were re-suspended between each aliquot.

Storage

The cryovials were transferred from Manufacturing Cleanroom—or—to a Room—where they were loaded into a LN2 Controlled Rate Freezer. The vials were frozen using a specified freezing profile to a temperature of negative 80-90° C. When complete the vials were transferred to a Quarantine LN2 Storage Dewar for storage until testing was complete. When testing was complete and the MCB was released by quality assurance the vials were transferred to the Released LN2 Storage Freezer until issued for preparation of Final Product Passage 1 (P1).

### Critical Parameters:

| | | | |
|---|---|---|---|
| Parameter | Low | Target | High |

General Equipment Conditions

| | | | | | | |
|---|---|---|---|---|---|---|
| Temperature | 36.5° | C. | 37.0° | C. | 37.5° | C. |

| | | | |
|---|---|---|---|
| CO₂ | 4% | 5% | 6% |
| Low O₂ | 4% | 5% | 6% |

P0 seed

| Seeding | N/A | | 6-15 cm TCDs for | | N/A | |
|---|---|---|---|---|---|---|
| | | | 2 eye globes, | | | |
| # of 15 cm TCD | N/A | | 6 | TCD | N/A | |
| Media at Seed | 15 | mL//TCD | 20 | mL//TCD | 25 | mL//TCD |
| Media at Change | 15 | mL/TCD | 20 | mL//TCD | 25 | mL//TCD |

| | | | | | |
|---|---|---|---|---|---|
| Change Media | ~2 | Day | 3 | Days | N/A |
| Duration | 6 | Days | 7 | Days | 8 |

| | | | |
|---|---|---|---|
| Harvest Parameter | N/A | islands of the small | N/A |
| | | epithelioid | |
| | | hexagonal cells | |

P1 Seed and expansion

| Seed Density in TCDs | N/A | | 8 × 10⁴/TCD | | N/A | |
|---|---|---|---|---|---|---|
| # of 9 cm TCDs | 10 | | 20 | | 40 | |
| Media at Seed in TCDs | N/A | | 10 | mL/TCD | N/A | |
| Media at Change in | N/A | | 10 | mL/TCD | N/A | |
| TCDs | | | | | | |
| Change Media | 2 | | 3 | Days | N/A | |
| Duration | 3 | Days | 4 | Days | 6 | Days |

| | | | |
|---|---|---|---|
| Harvest Parameter | 75% Confluence | 80% Confluence | 85% Confluence |
| Freeze in 1.8 mL Vial | N/A | 1 × 10\hat{ }6/Vial | N/A |
| # Vials (17 wk) | 50 | 100-150 | 250 |

Preparation of Final Product

The RPE cell production process continued with the aseptic processing of the Final Product in Manufacturing Cleanrooms.

Culture of P2 (from Frozen Vial 1×10⁶)

The process was initiated with the requisition of a released frozen MCB vial. The vial was issued by Materials Management and transferred from Room_to Manufacturing Cleanroom_with the use of a portable dewar. The vial was thawed, washed and seeded into 9 cm tissue culture dishes (TCDs) for expansion Passage 2 (P2). When the cells reached confluence they were harvested and further expanded in 636 cm² Corning Cell Culture Chambers to Passage 3 (P3). The stepwise procedure was as follows:

1. The cryovial was immersed in a 37° C. water bath without submerging the cap. The vial was gently swirled until a small ice crystal remains. Next the vial was removed and transferred to the Biosafety Cabinet (BSC). The cryovial was wiped with ethanol or isopropanol before opening. A pipette was used to transfer the 1 mL of cells into a new 50 mL centrifuge tube.

2. Next 5 mL of pre-warmed RPEGM was added dropwise while gently mixing the tube. This initial addition of media was performed slowly to reduce osmotic shock to the cells. Then 10 mL of 37° C. RPEGM was slowly added to the tube. The cell solution was gently resuspended by 10 ml pipette few times. The tube was centrifuged at 500 RCF for 5 minutes at room temperature. The supernatant was removed and the cells were re-suspended in 0.5 mL of 37° C. RPEGM.

3. The cells were mixed by gentle aspiration to ensure a homogeneous suspension. A cell count was performed using a hemacytometer. The cell suspension was diluted to a target concentration of 1×10⁵ cells/mL with RPEGM.

4. The required number of 9 cm TCDs was prepared by pipetting 9 mL of RPEGM into each TCD. Number of TCDs: total number of viable cells/1×10̂5.

5. The cells were aspirated and 1 mL of cell suspension was seeded via pipette into each TCD.

6. The TCDs were incubated at 37° C., 5% CO₂ and 5% O₂.

7. RPEGM was changed every other day after seeding.

8. The TCDs were monitored as required to check for confluence.

9. The culture was expanded for 3-5 days until the cells in TCDs reach a confluence of 80-85%.

Expansion to P3

After the culture had been expanded for 3-6 days and the TCDs had reached a confluence of 80-85%, the harvest and expansion was initiated. The cells were harvested and collected, counted, and then Passage 3(P3) was seeded into 636 cm² Culture Cell Corning Chambers. The stepwise procedure is as follows:

1. The media from each TCD was removed and discarded via pipette.

2. Next 7 mL of phosphate buffered solution (PBS) was added to each TCD via pipette. The TCDs were rocked back and forth to rinse the attached cell layer. The PBS from each TCD was removed and discarded via pipette.

3. Next 5 mL of TrypLE Express Solution was added to each TCD. The TCDs were rocked back and forth to ensure that the cells were saturated. The TCDs were incubated at 37° C., 5% CO₂ and 5% O₂ for a minimum of 10 minutes.

4. At the completion of incubation the TCDs were removed from the incubator. At least one TCD was observed under a microscope to ensure that a majority of the cells had detached.

5. Next 5 mL of RPEGM was added to each TCD via pipette. The dishes were washed by rinsing media over the TCD surface several times via pipette. When completed the cells were collected via pipette and transferred to the required number of 50 mL tube(s) (1 TCD per one tube). This step was repeated a second time to ensure that the majority of the cells had been collected.

6. The tubes were centrifuged at 500 RCF for 5 minutes at room temperature. At the completion of centrifugation the supernatant was discarded, the cells were re-suspended in 1 mL of RPEGM in each tube. The cells were aspirated and transferred to one collecting tube.

7. The cells were mixed by gentle aspiration to ensure a homogeneous suspension. A cell count was performed using a hemacytometer. The cell suspension was diluted to a target concentration of 2×10⁶/mL with RPEGM. Number of chambers: Total number of viable cells/2×10̂6

8. Required number of 636 cm₂ Culture Corning Chambers was prepared 0.100 mL of RPEGM was added into each chamber. Next, 1 ml of the cell suspension (2×10̂6) was added in to the same chamber.

9. An additional 50 mL of RPEGM was added to each chamber via pipette. A maximum of 5 chambers was seeded at one time, the cells were re-suspended between each seeding.

10. The chambers were incubated at 37° C., 5% CO₂ and 5% O₂.

11. The chambers were monitored as required to check for confluence. The media was changed every 2-3 days until a confluence of 80-85%. The media was removed and discarded. 150 mL of fresh RPEGM was added to each chamber via pipette.

12. The culture was expanded for 3-6 days until the chambers reached a confluence of 80-85%.

Harvest of P3

After the culture had been expanded for 3-6 days and the chambers had reached a confluence of 80%-85%, the harvest was initiated. The cells were harvested and collected, counted, re-suspended in freezing medium and 1 mL was aliquoted into 1.8 mL cryovials at a target concentration of 2×10⁶ cells/mL. The stepwise procedure was as follows:

1. The media from each chamber was removed and discarded by aspirating using a pipet into the waste container.

2. Next 50 mL of phosphate buffered saline (PBS) was added to each chamber via pipette. The chambers were rocked back and forth to rinse the attached cell layer. The PBS from each chamber was removed by aspirating using a pipet into the waste container.

3. 30 mL of TrypLE Express Solution was added to each chamber. The chambers were rocked back and forth to ensure that the cells were saturated. The chambers were incubated at 37° C., 5% CO₂ and 5% O₂ for a minimum of 10 minutes.

4. At the completion of incubation the chambers were removed from the incubator. At least one chamber was observed under a microscope to ensure that the majority of the cells had detached.

5. 30 mL of HBSS was added to each chamber via pipette. The chambers were washed by tipping and gently rocked back and forth. When complete, the cell suspension was aspirated using the pipet into the required number of 225 mL tube(s) (one tube per chamber).

6. This step was repeated a second time to ensure that the majority of the cells had been collected. At least one chamber was checked to ensure the majority of the cells have been removed.

7. The 225 mL tubes were centrifuged at 500 RCF for 5 minutes at room temperature. At the completion of centrifugation the supernatant was discarded and the cells re-suspended in 5-10 mL of HBSS in each tube. The cells from each 225 mL tube were aspirated and transferred to one collecting tube.

8. The cells were mixed by gentle aspiration to ensure a homogeneous suspension. A cell count was performed using a hemacytometer.

9. The required amount of freezing medium was prepared: total number of cells/2×10̂6.

10. Next, the tube was centrifuged at 500 RCF for 5 minutes at room temperature.

The supernatant was removed and discarded from the tube.

11. The cell suspension in the tube was diluted in freezing media to a target concentration of 2×10⁶ cells/ml. The cells were aspirated and 1 mL was aliquotted into 1.8 mL cryovials utilizing a 5 mL pipette. A maximum of 5 cryovials was aliquoted at one time, the cells were re-suspended between each aliquot.

Storage

The cryovials were transferred from Manufacturing Cleanroom_or_to Room_where they were loaded into a LN2 Controlled Rate Freezer. The vials were frozen using a specified freezing profile to a temperature of negative 80-90° C. When complete the vials were transferred to a Released LN2 Storage Freezer for storage until testing was complete and the Final Product (P4) was Released by Quality Assurance for shipment or final formulation.

### Critical Parameters:

| | | | |
|---|---|---|---|
| Parameter | Low | Target | High |

General Equipment Conditions

| | | | | | | |
|---|---|---|---|---|---|---|
| Temperature | 36.5° | C. | 37.0° | C. | 37.5° | C. |

| | | | |
|---|---|---|---|
| CO₂ | 4% | 5% | 6% |
| Low O₂ | 4% | 5% | 6% |

P0 seed

| Seeding | N/A | | 6-15 cm TCDs for | | N/A | |
|---|---|---|---|---|---|---|
| | | | 2 eye globes, | | | |
| # of 15 cm TCD | N/A | | 6 | TCD | N/A | |
| Media at Seed | 15 | mL//TCD | 20 | mL//TCD | 25 | mL//TCD |
| Media at Change | 15 | mL/TCD | 20 | mL//TCD | 25 | mL//TCD |

| | | | | | |
|---|---|---|---|---|---|
| Change Media | ~2 | Day | 3 | Days | N/A |
| Duration | 6 | Days | 7 | Days | 8 |

| | | | |
|---|---|---|---|
| Harvest Parameter | N/A | islands of the small | N/A |
| | | epithelioid | |
| | | hexagonal cells | |

P1 Seed and expansion

| Seed Density in TCDs | N/A | | 8 × 10⁴/TCD | | N/A | |
|---|---|---|---|---|---|---|
| # of 9 cm TCDs | 10 | | 20 | | 40 | |
| Media at Seed in TCDs | N/A | | 10 mL/TCD | | N/A | |
| Media at Change in | N/A | | 10 mL/TCD | | N/A | |
| TCDs | | | | | | |
| Change Media | 2 | | 3 | Days | N/A | |
| Duration | 3 | Days | 4 | Days | 6 | Days |

| | | | |
|---|---|---|---|
| Harvest Parameter | 75% Confluence | 80% Confluence | 85% Confluence |
| Freeze in 1.8 mL Vial | N/A | 1 × 10\hat{ }6/Vial | N/A |
| # Vials (17 wk) | 50 | 100-150 | 250 |

RPE Cells Biomarker Characterization

Reagents

Monoclonal Anti-Cytokeratin, pan antibody produced in mouse (Cat # P2871, Sigma-Aldrich) 2 mg/ml, 7.5 μl-15 μl/mL, dilution 1 μl-2 μg).

Antibody Rabbit anti ZO-1 (Cat #61-7300, Invitrogen), 0.25 mg/ml in PBS, Invitrogen, working solution 2-5 mcg/ml, 8 μl-20 μl, 10-0.25 μg).

RPE 65 Antibody (mouse monoclonal) (Cat # NB 100-355, Novus Biologicals) 1/mg/ml, 0.1 ml, dilution 1:5000-1:10000), 1 μL-1 μg

Fibroblasts MOUSE ANTI-Human Monoclonal Antibody (LS-C66110,-LSBio, 1 μL-0.1 μg recommended dilution 1:100)

CD31/PECAM1 Rabbit Polyclonal Antibody (NBP1-71663, Novus Biologicals, 0.6 μg/1 μl, recommended dilution for flow 1:1000)

Fluorescent Anti-Mouse IgG (Cat # FI-2000, lot # X0521 Vector Laboratories, Maximum excitation 490-500 nm, maximum emission 510-520 nm, 1.5 mg/mL active conjugate, recommended concentration range for use is 5-20 μg/mL, 3.3-13.3 μl in 1 ml, 1 μl-1.5 μg)

Fluorescent Anti-Rabbit IgG (Cat # FI-1000, lot # X0521 Vector Laboratories, Maximum excitation 490-500 nm, maximum emission 510-520 nm, 1.5 mg/mL active conjugate, recommended concentration range for use is 5-20 μg/mL, 3.3-13.3 μl in 1 ml, 1 μl-1.5 μg)

BD Cytofix/Cytoperm Fixation/Permeabilization Kit (Cat. 554714, BD biosciences)

Staining protocol for performance markers.

1. Obtained required amount of vials for testing each lots. Thawed the cryovials in a 37° C. water bath. Transferred cells in 50 mL tube and slowly added 15 mL of PBS.

2. Centrifuged cells at 550 RCF for 5 minutes at room temperature.

3. Decanted the supernatant and resuspended the cell pellet in PBS to have a final cell concentration of 10-15×10⁶ per 20 ml. Mixed cells by gentle aspiration to ensure a homogeneous suspension. Perform cell count as per TM-008. Record the cell count and viability results in form_{—}

4. Centrifuged the cells at 500 RCF for 5 minutes at room temperature, decanted supernatant.

5. Re-suspended cells in 5 mL of Cytofix Solution. Mixed cells by gentle aspiration to ensure a homogeneous suspension.

6. Incubated at 4° C. for 20 minutes in dark refrigerator on a rotator (or on a rotator in covered ice bucket at room temperature). Cells may get more buoyant after fixation.

7. Centrifuged the cells at 500 RCF for 5 minutes at room temperature, decanted supernatant.

8. Re-suspended cells in 10 mL of 1× CytoPerm/Wash buffer. Mixed cells well by gentle aspiration to ensure a homogeneous suspension. Incubated at 4° C. for 5 minutes in dark refrigerator on a rotator (or on a rotator in covered ice bucket at room temperature).

| Parameter | Low | Target | High |
|---|---|---|---|
| Number of RPE | 8 × 10⁵ | 1 × 10⁶ | 1.2 × 10⁶ |
| cells per mL | | | |

9. Centrifuged the cells at 500 RCF for 5 minutes at room temperature, decanted supernatant.

10. Re-suspended cells in 10 mL of 1× CytoPerm/Wash buffer. Mixed cells well by gentle aspiration to ensure a homogeneous suspension. Incubated on a rotator in a dark refrigerator or on ice.

11. Centrifuged the cells at 500 RCF for 5 minutes at room temperature, decanted supernatant.

12. Re-suspend cells in 10 mL of Staining (BSA) buffer. Mixed cells by gentle aspiration to ensure a homogeneous suspension. Incubated on a rotator in a dark refrigerator or on ice.

13. Centrifuged the cells at 500 RCF for 5 minutes at room temperature.

14. Re-suspended cells in 10 mL of PBS. Mixed cells by gentle aspiration to ensure a homogeneous suspension. Proceeded with staining next day or they can be stained immediately.

Note: At this point cells may be stored for up to one week at 2-8° C. in the dark.

15. Labeled 1.8 L tubes. as follows:

a. fixed RPE cells as negative controls with unlabeled anti cytokine primary antibody

b. fixed RPE cells as negative controls with fluorochrome labeled secondary antibody

c. fixed RPE cells as test samples with primary and fluorochrome labeled anticytokine antibodies.

Negative Controls:

Primary Antibody Negative Control

| | | | | |
|---|---|---|---|---|
| .Primary | Pan cytokeratin | ZO-1 | RPE65 | Fba |
| antibody | 2 μL-4 μg | 2 μL-0.5 μg | 2 μl-2 μg | 1 μL-0.1 μg |
| Secondary | N/A | N/A | N/A | N/A |
| antibody | | | | |

Secondary Antibody Negative Control

| | | |
|---|---|---|
| Primary | N/A | N/A |
| antibody | | |
| Secondary | Fluorescent | Fluorescent |
| antibody | Anti-Mouse | Anti-Rabbit |
| | IgG | IgG |
| | 1 μl-1.5 μg | 1 μl-1.5 μg |

Test Samples for Performance Markers

| | | | | |
|---|---|---|---|---|
| Primary | Pan cytokeratin | ZO-1 | RPE65 | Fba |
| antibody | 2 μL-4 μg | 2 μL-0.5 μg | 2 μl-2 μg | 1 μL-0.1 μg |
| Secondary | Fluorescent | Fluorescent | Fluorescent | Fluorescent |
| antibody | Anti-Mouse | Anti-Rabbit | Anti-Mouse | Anti-Mouse |
| | IgG | IgG | IgG | IgG |
| | 1 μl-1.5 μg | 1 μl-1.5 μg | 1 μl-1.5 μg | 1 μl-1.5 μg |

16. Centrifuged tubes at 500 RCF for 5 minutes at room temperature, decanted supernatant with pipet without disturbing a pellet.

17. Added PBS to dilute to a target concentration 1×10⁶ per 100 μL. Mixed the cell suspension well with a pipet.

18. Added un-conjugated primary antibodies to the designated negative primary controls and tested tubes.

19. Vortexed and incubated for 60 minutes in 4° C. refrigerator on a rotator (or on a rotator in covered ice bucket at room temperature).

20. Centrifuged tubes at 500 RCF for 5 minutes at room temperature, decanted supernatant with pipet without disturbing a pellet.

21. Added 1.0 mL of PBS to each tube to wash off excess antibody. Mixed the cell suspension gently 8-10 times with pipet.

22. Centrifuged tubes at 500 RCF for 5 minutes at room temperature, decanted supernatant with pipet without disturbing a pellet.

23. Next, added 500 μL of PBS, mixed cells well.

24. Added 10 of fluorochrome—conjugated secondary antibody to all tubes except the negative controls with primary antibodies.

25. Vortexed and incubate for 60 minutes in 4° C. refrigerator on a rotator (or on a rotator in covered ice bucket at room temperature).

26. Centrifuged tubes at 500 RCF for 5 minutes at room temperature, decanted supernatant with pipet without disturbing a pellet.

27. Added 1.0 mL of PBS to each tube to wash off excess antibody. Mixed the cell suspension gently 8-10 times with pipet.

28. Centrifuged tubes at 550 RCF for 5 minutes at room temperature, decanted supernatant with pipet without disturbing a pellet.

29. Re-suspended cells in 200 μL PBS

Characterizing Polarized RPE Monolayers.

Reagents

Human Fibronectin (1 mg, Cat #354008, BD)

Antibody Rabbit anti ZO-1 (Cat #61-7300, dilution 1:100).

Anti-NA+/K+ATPase a-1, clone C464, (monoclonal Antibody, Cat #05-369, Lot #05369, Millipore dilution 1:100)

Phosphate Buffered Saline (PBS) (Gibco, Catalog #20012)

20% Formaldehyde Solution EM Grade, Catalog#15713, Electron Microscopy)

BSA fraction V (Gibco, Catalog #15260)

Triton X-100, (Sigma-Aldrich, Catalog# T 9284-100 ml)

DAPI (4,6 diamidino-2-phenilndole) solution) (Invitrogen, Catalog# D3571, lot#28870W)

Fluorescein anti-mouse IgG, FI-2000, green, Vector

Fluorescein anti-rabbit IgG, FI-2000, green, Vector

2% Formaldehyde Fixative Solution (10 ml)

| Name | Quantity | | Manufacturer |
|---|---|---|---|
| 20% Formaldehyde Solution | 1 | mL | Electron Microscopy |
| PBS | 9 | mL | Gibco |

Permiabilization solution: 0.2% Triton X-100 (10 ml)

| Name | Quantity | | Manufacturer |
|---|---|---|---|
| Triton X-100 | 20 | μL | Sigma-Aldrich |
| PBS | 10 | mL | Gibco |

Blocking buffer: 1% BSA/PBS (10 ml)

| Name | Quantity | | Manufacturer |
|---|---|---|---|
| BSA fraction V | 100 | μL | Gibco |
| PBS | 10 | mL | Gibco |

Dapi Nuclear Stain Solution:

Preparation: added 1 μl of 14 mM stock for every 5 ml of PBS. Stored any unused DAPI at 2-8° C., wrapped in aluminum foil.

RPE Growth Media (100 mL)

| Name | Quantity | | Manufacturer |
|---|---|---|---|
| MEM | 462 | mL | Sigma |
| NEAA | 1 | mL | Sigma |
| GlutaMax | 1 | mL | Invitrogen |
| bFGF | 2 | μg | PeproTech |
| Heparin Sodium InJ. | 600 | μl | APP Pharmaceutical |
| 1,000 USP/mL | | | |
| FBS | 5 | mL | Thermo-Sci |
| B-27 supplement(50X) | 2 | mL | Gibco |
| minus vitamin A | | | |

Materials

Human Fetal RPE Cells

Transwell (Corning Costar, 3460-Clear, 0.4 μm pores, 12 mm inner diameter inserts, polyester membrane polystyrene plates)

Equipment

37 C 5% CO2, 20% O2 incubator

Inverted Microscope

Fluorescent Microscope

1. equilibrated vial to room temperature.

2. Re-suspended in one milliliter sterile distilled water.

3. Allowed 30 minutes for material to go into solution. Did not agitate or swirl.

4. Following reconstitution in distilled water, diluted to concentration 50 μg/ml with Ca++, Mg++ free PBS by adding 19 ml of PBs to obtain total volume 20 ml.

5. Added 0.3 ml of the fibronectin solution and incubated coated filters for 1 hour at room temperature.

6. Aspirated remaining material.

7. Rinsed plates carefully with distilled water. Avoided scrapping bottom surface.

8. Plates were ready to use.

Note: If entire amount of material was not used immediately, transferred in to appropriate aliquots and store at −20 C for 1 month. Avoid multiple freeze thaws.

Culture of Polarized RPE Monolayers

1. Prepared the Transwell filters by coating the upper compartment with 300 μl of fibronectin.

| | | | |
|---|---|---|---|
| Control | ZO-1 | ZO-1 | ZO-1 |
| Transwell | Transwell | Transwell | Transwell |
| inserts | inserts | inserts | inserts |
| Control | Na/K ATPase | Na/K ATPase | Na/K ATPase |
| Transwell | Transwell | Transwell | Transwell |
| inserts | inserts | inserts | inserts |

2. Thawed cells as described in Process Description for RPE cells.

3. Re-suspended the cells in RPEGM containing 5% serum. Mixed the cells well by pipetting up and down to obtain a homogeneous cell suspension.

4. Seeded onto the Transwells at a density of 1×10̂5 cells per transwell (600 μl) and introduced 1.5 ml of 5% FBS containing media to the lower compartment. Do not add cells to control inserts.

5. After overnight incubation with RPEGM containing 5% FBS, changed ½ culture media to 1% FBS RPE differentiation media (RPEDM). Removed 300 μl of culture medium from the top chamber and added 300 μl of 1% FBS RPE. Followed by removal 750 μl of culture medium from the bottom chamber and addition 750 μl of 1% FBS RPE.

6. Next, after overnight incubation with mixed media, changed the media to 1% FBS containing RPEDM. Added 600 μl of medium first to the top chamber of Transwell followed by 1.5 ml of medium to the bottom chamber. Changed the medium every 3-4 days with 1% FBS RPEDM.

Measuring Transepithelial Resistance

Procedure

1. Removed culture media from each of the inserts and wells and washed twice with 2 ml of PBS. Added 0.5 mL of the solution to the top and 1.5 mL to the bottom chamber.

2. Next, added 0.5 mL of cold 2% Formaldehyde Fixative Solution to the top and 1.5 mL to the bottom chamber. Incubated for 30 minutes at room temperature.

3. Removed the fixative and permeabilize the RPE cells. Incubated cells with 0.2% Triton X-100 for 30 min at room temperature. Added 0.5 mL of the solution to the top and 1.5 mL to the bottom chamber.

4. Discarded permeabilization solution.

5. Washed the Transwell inserts with 5 ml of PBS (RT) for two times.

6. Blocked specimens in 5 mL of 1% BSA/PBS solution for 1 hr at room temperature.

7. Removed blocking buffer. No rinsing was necessary.

8. Incubated with antibodies at 4° C. diluted in 1% BSA/PBS solution. Added 0.3 ml of the solution to the top and 0.5 ml to the bottom chamber) overnight (or for 1 hour at room temperature). Antibodies were diluted as described in the table below.

| Antibody | Dilution |
|---|---|
| Antibody Rabbit anti-ZO-1 | 1:100 |
| Antibody Anti-Na+/K+ ATPase | 1:100 |

9. A negative control was performed using PBS containing 1% BSA/PBS with no primary antibody.

10. Removed the primary antibodies and washed three times (each 5 min) with 5 ml of PBS and incubated with FITC-conjugated secondary antibodies (see step 9) for 40 min at RT in the dark (dilution 1:100).

11. Discarded the secondary antibodies and wash membranes two times with PBS (see step 6).

12. Counterstained with diluted DAPI solution (100 ng/ml) for 15 minutes at room temperature. Added 0.5 mL of the solution to the top and 1.5 mL to the bottom chamber.

13. Rinsed once with 5 ml PBS and once with 5 ml distilled water. Visualized using a fluorescence microscope.

What is claimed is:

1. A method for differentiating retinal pigmented epithelium (RPE) into neuronal-like cells comprising the step of culturing RPE cells in vitro under low oxygen.

2. The method of claim 1 wherein said culturing comprises supplementation with bFGF.

3. The method of claim 1 wherein said culturing excludes non-hypoxic conditions.
